(19) United States
(12) Patent Application Publication
(10) Pub. No.: US 2014/0296937 A1
(43) Pub. Date: Oct. 2, 2014

(54) PULSED INFRARED MODULATION OF A PHOTOVOLTAIC CELL COCHLEAR IMPLANT FOR THE MULTICHANNEL ELECTRICAL DEPOLARIZATION OF SPIRAL GANGLION CELLS TO ACHIEVE SOUND PERCEPT

(71) Applicant: INFRARED COCHLEAR PARTNERS, LLC, Savannah, GA (US)

(72) Inventors: Manning M. GOLDSMITH, Savannah, GA (US); Byron L. BOYLSTON, Louisville, KY (US); Robert T. STONE, Sunnyvale, CA (US)

(73) Assignee: INFRARED COCHLEAR PARTNERS, LLC, Savannah, GA (US)

(21) Appl. No.: 14/243,297

(22) Filed: Apr. 2, 2014

Related U.S. Application Data
(60) Provisional application No. 61/807,535, filed on Apr. 2, 2013.

(51) Int. Cl. A61N 1/36

(52) U.S. Cl. CPC A61N 1/36032
USPC 607/57

(57) ABSTRACT

A hearing assistance device for use with respect to a mastoid cavity and an ear canal with a tympanic membrane, external ear canal bone, incudo-stapedial joint of an ossicular chain and a cochlea having a round window membrane. The device includes a removable unit with a microphone to convert a received sound into electrical sound signals, and includes a digital audio signal processor to process the electrical sound signals into envelopes of the electrical sound signals, and includes an infrared transmitter to transmit the envelopes of sound signals as packages of infrared light pulses. The hearing assistance device also includes an implant unit with a photovoltaic cell to receive the packages of light pulses and demodulate the packages of light pulses into electrical signals, and at least one active electrode that delivers the demodulated electrical signals to the cochlea.

### CROSS-REFERENCE TO RELATED APPLICATION

This application claims the benefit of U.S. Provisional Patent Application Ser. No. 61/807,535 filed Apr. 2, 2013, the entirety of which is hereby incorporated by reference herein.

### TECHNICAL FIELD

The present invention is directed to the field of cochlear implants for patients with hearing impairment and/or tinnitus.

### BACKGROUND

In its naturally occurring anatomy, the human ear consists of an auditory canal terminating externally in a pinna and internally at the ear drum or tympanic membrane, a series of three small bones in proximity to the ear drum where the ear drum and three bones are located in the middle ear, a cochlea and plural auditory nerves leading to the brain. In natural operation, sound waves enter the ear through the pinna where pressure differences between successive compressions and rarefactions set the eardrum vibrating. These vibrations are amplified by the middle ear mechanism and transferred to the cochlea, where they are transduced to electrical signals. The inner ear structures responsible for this transductive function are known as hair cells. The ability of the human ear to recognize differing frequencies is in part attributed to the location along the basilar membrane at which the travelling sound wave stimulates the individual hair cells. The electrical currents which are produced in response to the mechanical stimulation by sound are known as cochlear microphonics. The sound signal is tonotopically sorted into its component frequencies by the membranous structures of the inner ear, such that the higher frequencies are sorted in the basal regions and the lower frequencies are sorted more distally toward the apical portion. These signals then travel along the auditory nerves to the brain where they are perceived as sound.

An estimated 200-300 million people have various patterns of severe sensorineural hearing loss, which is imperfectly rehabilitated via hearing aids. For these types of hearing loss, mere amplification is ineffective because the cochlea cannot perform its transductive function of converting the mechanical energy of sound to the electrical current ultimately perceived as sound by the brain. When the hair cells are sufficiently damaged as in the above mentioned scenarios no amount of amplification will be effective.

Cochlear implants assist these profoundly deaf. These cochlear implants are, in effect, bionic ears that replace the lost cochlear microphonic with an electrical current which is the precise analog of sound. Currently available cochlear implants are expensive, highly complex devices that must be surgically introduced via a complicated and, for the average otolaryngologist, risky procedure under general anesthesia known as the facial recess mastoidectomy. The prohibitive price and impractical complexity deter accessibility to the vast majority of the global deaf population. And, the average otologist in the developing countries of the world typically does not have the sophistication, expertise and equipment to confidently undertake the facial recess mastoidectomy in order to introduce the internal component of the multichannel systems.

An example Transcanal Cochlear Implant System (U.S. Pat. No. 7,120,501, incorporated herein by reference for all purposes) describes a single electrode cochlear implant which is passively powered by an electromagnetic field modulation between an external coil from a hearing aid in the ear canal to an internal coil in an implanted portion within the middle ear. However, this system is only capable of delivering an analog audio signal to the cochlea through a single channel.

As a result, there exists a need for an improved system and/or method to assist the profoundly deaf, who are unable to hear sound through mere amplification.

### SUMMARY

The described technology relates to an infrared modulation system for a transcanal cochlear implant that is operable with a minimum of surgical intrusion and can be installed at a physician's office under local anesthesia. The infrared modulation system replaces the transducer function of the cochlea when insufficient inner hair cells are present within the cochlea to perform this function. Infrared color coded (or wavelength encoded) pulses of light permit an active electrode to deliver envelopes of varying frequencies tonotopically to the corresponding frequency-tuned spiral ganglion hair cells of the cochlear modiolus in the form of discreet pulsed digital electrical fields through multiple channel bands, in order for the patient to better discriminate pitch from the resultant signal. The implant portion can be located in the middle ear or the mastoid cavity.

According to another aspect, the described technology relates to a hearing assistance device for use with respect to a mastoid cavity, and an ear canal with a tympanic membrane, external ear canal bone, incudo-stapedial joint of an ossicular chain and a cochlea having a round window membrane. The hearing assistance device includes a removable unit with a microphone to convert a received sound into electrical sound signals. The removable unit also includes a digital audio signal processor to process the electrical sound signals into envelopes of the electrical sound signals. The removable unit also includes an infrared transmitter to transmit the envelopes of sound signals as packages of infrared light pulses. The hearing assistance device also includes an implant unit with a photovoltaic cell to receive the packages of light pulses and demodulate the packages of light pulses into electrical signals, and at least one active electrode that delivers the demodulated electrical signals to the cochlea.

According to still another aspect, the described technology relates to a hearing assistance device for use with respect to a mastoid canal, and an ear canal with a tympanic membrane, bone and a cochlea having a round window membrane. The hearing assistance device includes a removable unit with a microphone to convert a received sound into electrical sound signals. The removable unit also includes a digital audio signal processor to process the electrical sound signals into envelopes of the electrical sound signals, and an infrared transmitter to transmit the envelopes of sound signals as packages of infrared light pulses. The hearing assistance device also includes an implant unit with a photovoltaic cell to receive the packages of light pulses and demodulate the packages of light pulses into electrical signals. The hearing device further includes a pulse transformer that isolates the electrical signals from the photovoltaic cell into encoded frequency envelopes and outputs an electrical current to the cochlea as interleaved pulsed electrical fields.

According to still another aspect, the described technology relates to a method for delivering an audio sound signal to the nerves extending from a cochlea. The method includes converting a received audio sound signal into a plurality of electrical signals, and processing the plurality of electrical signals into envelopes of electrical sound signals. The method also includes transmitting the envelopes of sound signals as packages of infrared light pulses, and demodulating the packages of infrared light pulses into electrical signals. The method further includes isolating the demodulated electrical signals into encoded frequency envelopes, and outputting the encoded frequency envelopes as interleaved pulsed electrical fields within the cochlea.

When taken in conjunction with the accompanying drawings and the appended claims, other features and advantages of the present invention become apparent upon reading the following detailed description of embodiments of the invention.

### BRIEF DESCRIPTION OF THE DRAWINGS

Example embodiments of the invention are illustrated in the drawings in which like reference characters designate the same or similar parts throughout the figures of which:

FIG. 1 illustrates a sectional diagram of a human ear incorporating the present invention according to a first example embodiment; and

FIG. 2 illustrates a top level block diagram of the present invention of FIG. 1 according to a first exemplary embodiment.

FIG. 3 illustrates a top level block diagram of the present invention of FIG. 1 according to a second exemplary embodiment.

FIG. 4 illustrates a top level block diagram of the present invention of FIG. 1 according to a third exemplary embodiment.

FIG. 5 illustrates a top level block diagram of the present invention of FIG. 1 according to a fourth exemplary embodiment.

FIG. 6A illustrates a side view of a sectional diagram of a human ear incorporating the present invention according to a second example embodiment.

FIG. 6B illustrates a rear view of the sectional diagram and second example embodiment shown in FIG. 6B.

FIG. 7 illustrates a sectional diagram of a human ear incorporating the present invention according to a third example embodiment.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

An infrared modulation system for a multichannel transcanal cochlear implant is presently contemplated that is operable with a minimum of surgical intrusion and can be installed at a physician's office under local anesthesia. The infrared modulation system replaces the transducer function of the cochlea when insufficient inner hair cells are present within the cochlea to perform this function. Infrared color coded (or wavelength encoded) pulses of light permit an active electrode(s) to deliver envelopes of varying frequencies tonotopically to the corresponding frequency-tuned spiral ganglion hair cells of the cochlear modiolus in the form of discreet pulsed digital electrical fields through multiple channel bands, in order for the patient to better discriminate pitch from the resultant signal.

The infrared modulation system can be configured with the external components placed in a package similar to a commercially available ITE (in the ear hearing processor) or a commercially available BTE (behind the ear processor). The infrared modulation system external components can contain a digital signal processor (DSP), which processes a speech signal, separating spectral envelopes of sound through bandpass filters. The frequency envelopes are transmitted from distinct light emitting diodes (LEDS) positioned on the distal end of the processor, within the ear canal and near the tympanic membrane. The digital pulses are transmitted at high pulse rates across the tympanic membrane, where they are received by separate tandem layers of a quantum dot photovoltaic cell configured to fit in the middle ear space. The quantum dot photovoltaic cell is configured such that by varying the size of the quantum dots, specific band gaps can be “tuned” or matched to certain wavelengths of the transmitted infrared signals, thereby separating the encoded frequency bands into channels. The encoded frequency bands are demodulated and delivered as direct current electrical pulses to a transformer, which isolates the direct current bias of the signal and outputs a bipolar electrical current to the multiple channel bands of an active electrode inserted into the cochlea. These channeled electrical currents are pulsed at high rates in an interleaved fashion to avoid channel interactions.

The frequency envelopes can alternatively be transmitted by methods understood to persons skilled in the art, for example as radiofrequency or electromagnetic inductive energy, as explained in U.S. Pat. No. 7,120,501, incorporated herein by reference for all purposes.

The infrared modulation transmission system is MRI compatible because it is functional without magnets.

Alternatively, the infrared modulated cochlear implant can also be combined with an existing hearing aid for the delivery of combined Electrical and Acoustic Stimulation (EAS). In example EAS, amplified acoustic signals are delivered to improve residual hearing in the low to mid frequency spectra, while electrical signals are conveyed to rehabilitate severe to profound high frequency loss where hair cell function is absent. In a preferred embodiment EAS sound processor configuration (e.g., ITE or BTE), light emitting diodes (LEDs) surround a central air core through which the amplified acoustic signal is transmitted. The LEDs transmit the high frequency electrical signal through pulsed IR modulation of the implanted internal photovoltaic cell and multichannel electrode inserted into the cochlea. The amplified low to mid frequency acoustic signals are transmitted to the tympanic membrane whereupon they are further transmitted through the ossicular chain to the cochlea. A digital signal processor (DSP) within the ITE or BTE processes both the acoustic and electrical signals and adjusts phase differences for delivery from a single device. Alternative example embodiment EAS systems can function with two separate devices, for example a hearing aid for delivery of the acoustic signal and a separate transmastoid cochlear implant for delivery of the electrical signal.

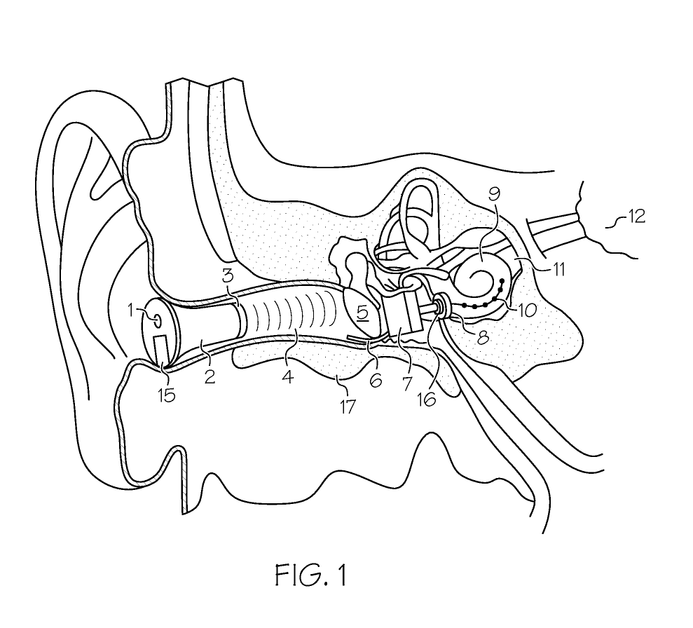

FIG. 1 depicts an example embodiment of the infrared modulation system installed within the cochlea 9 of an external auditory canal 17. As depicted, the infrared modulation system includes a removable processor unit 2 that fits within the auditory canal 17, for example with a friction fit. The removable processor unit 2 is positioned within the auditory canal 17 exterior to the anatomical tympanic membrane 5, or “ear drum.” The depicted removable processor unit 2 has at least one microphone 1, a power source 15 and an infrared transmitter 3. The power source 15 can be a battery and the infrared transmitter 3 can be a light emitting diode (LED). The microphone 1 and infrared transmitter 3 receive power from the power source 15.

The infrared modulation system also includes a surgically-implanted unit 7 positioned within the auditory canal 17 on the opposite side of the tympanic membrane 5 from the removable processor unit 2. The surgically-implanted unit 7 includes a ground electrode 6, and a multichannel active electrode 10. The active electrode 10 is inserted into the anatomical cochlea 9 through the anatomical round window membrane 8. The ground electrode 6 is inserted into a groove that has been surgically drilled into the anatomical bone of the external auditory canal 17 or secured/attached to the incudo-stapedial joint of the ossicular chain.

The distance that the distal tip of the electrode 10 can be inserted through the round window membrane 8 and into the cochlea 9 can be limited by a mechanical stop 16 integrally secured at a location along the electrode. As depicted, the mechanical stop 16 can have a circumferential shape extending radially from the electrode 8. The mechanical stop 16 extends to cover the opening of the round window membrane 8. The mechanical stop 16 can be installed in the round window membrane 8 opening by a surgeon, for example as described in U.S. patent application Ser. No. 12/897,903 filed on Oct. 5, 2010, incorporated by reference herein for all purposes. When installed in the round window membrane 8 opening, the mechanical stop 16 fixes the location of the surgically implanted unit 7, controls the insertion depth of the electrode 10 and helps seal the cochlea 9.

As depicted in FIGS. 1-5, the removable processor unit 2 can include a digital audio signal processor 14 electrically connected between the microphone 1 and the infrared transmitter 3. The microphone 1 detects an audio sound signal exposed to the ear. The audio sound signal detected by the microphone 1 is processed by the digital audio signal processor 14. The digital audio signal processor 14 digitally reformats the audio sound signal and encodes frequency envelopes of the audio sound signal into pulsed color coded packages, for example packages varying by their wavelength between 400 nm violet and 700 nm red. The pulsed wavelength encoded packages are sent by the infrared transmitter 3 as an infrared pulse 4 across the tympanic membrane 5. The infrared pulse 4 is received by the surgically implanted unit 7. The surgically implanted unit 7 demodulates the infrared pulse 4 into an electrical signal, for example with a demodulator circuit. The surgically implanted unit 7 then delivers this electrical signal through the electrode(s) 10 to the human brain 12 via the nerves 11 extending from the cochlea 9.

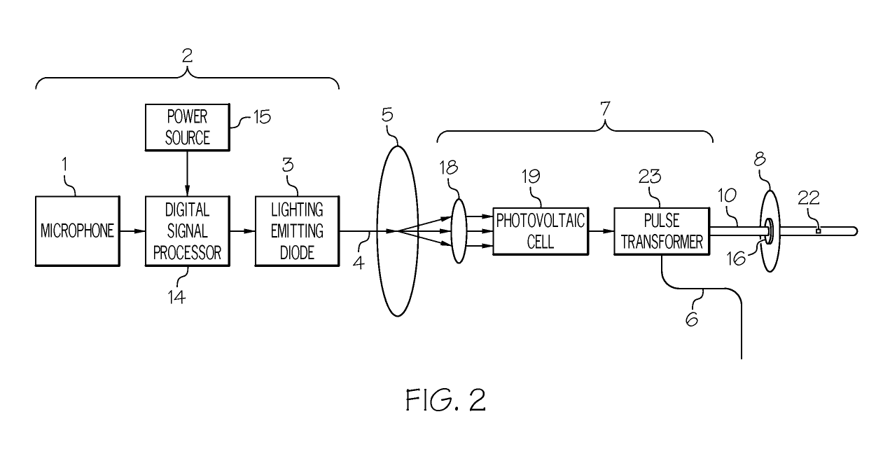

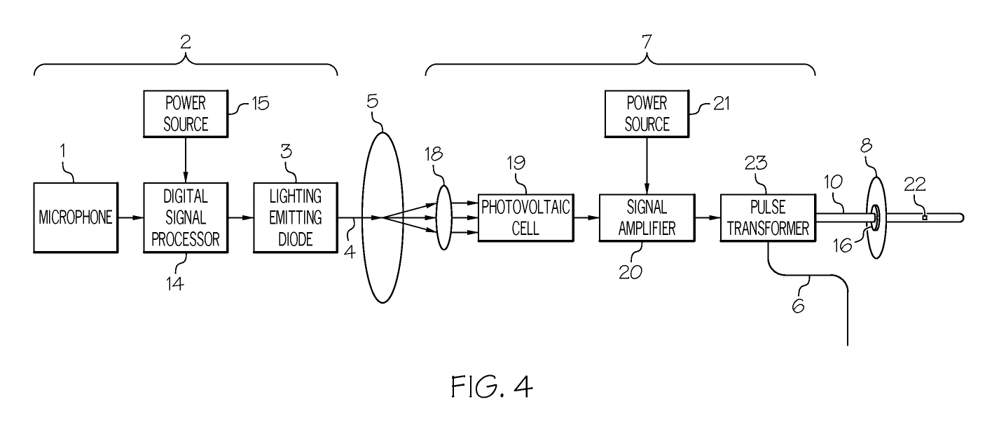

As depicted in FIGS. 2 and 4, the surgically implanted unit 7 can include an optical lens 18 preferably of convex shape, a photovoltaic cell 19 and a pulse transformer 23, from which the ground electrode 6 extends. The optical lens 18 can be constructed to receive and/or function with pulsed infrared light frequencies corresponding to an acceptable range of audio frequencies, for example between about 125 Hz and about 8,500 Hz, preferably between about 250 Hz and about 4,000 Hz, more preferably between about 500 Hz and about 2,000 Hz, and most preferably about 1,000 Hz, and excluding light frequencies outside of this range. The photovoltaic cell 19 is preferably a quantum dot photovoltaic cell and receives the infrared pulse 4 from the transmitter 3. Preferably, the photovoltaic cell 19 requires no connection to a power source. The operable distance between the infrared transmitter 3 to the photovoltaic cell 19 can vary, thus allowing effective transmission over different distances. The optical lens 18 helps correct for an optical diffusing effect of the pulse 4 caused by passage through the tympanic membrane 5. Through physically understood optical effects, the optical lens 18 increases the amount (e.g., intensity and/or surface area) of the transmitted light pulse 4 that is directed toward the photovoltaic cell 19. The photovoltaic cell 19 passively demodulates the color encoded pulse 4 and delivers the demodulated pulse 4 to the pulse transformer 23 as direct current electrical pulses. The pulse transformer 23 isolates the direct current bias of the signal into encoded frequency envelopes and outputs a bipolar electrical current through the electrode 10 as interleaved pulsed multichannel electrical fields. The distal end of the electrode 10 preferably includes a multi channel band 22 through which the interleaved pulsed electrical fields are delivered as an analog multichannel electrical signal to the cochlea nerves 11. The ground electrode 6 provides an electrical signal return path by completing the circuit for the electrical current delivered to the spiral ganglion cells as the current returns through the cochlear vasculature and connective tissues. The body tissue provides an electrical conduction path from the tip of the active electrode 10 to the ground electrode 6.

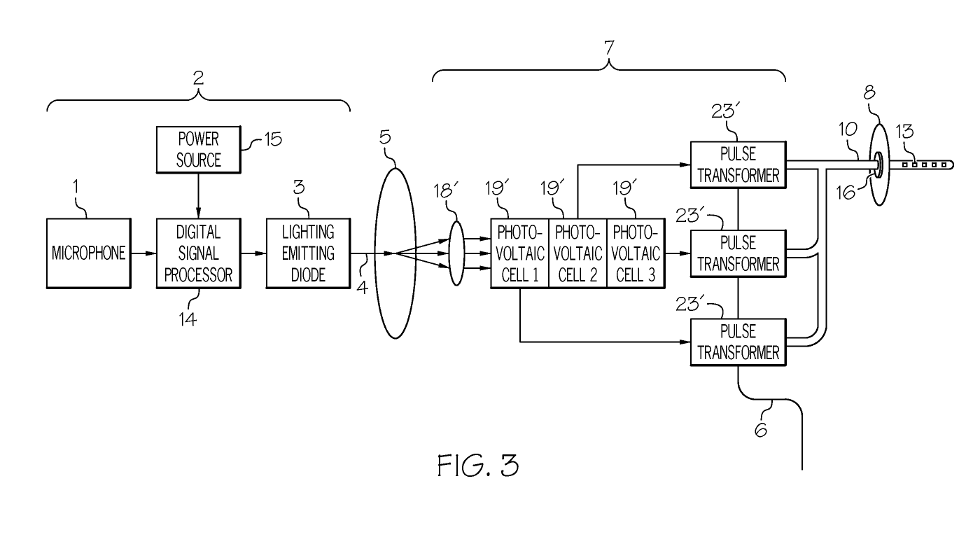

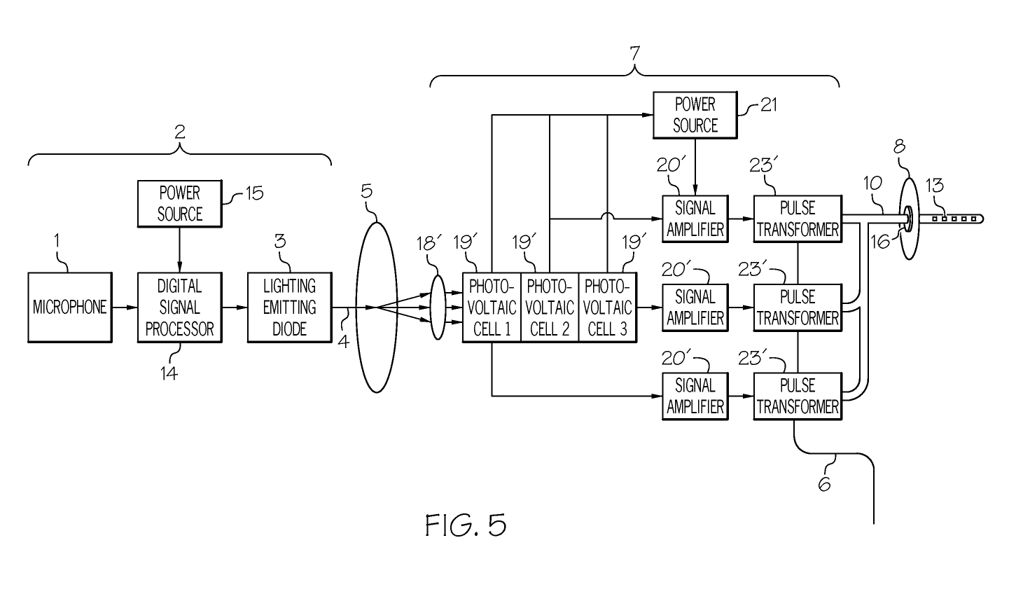

As depicted in FIGS. 3 and 5, the surgically implanted unit 7 can alternatively include at least one optical lens 18′ preferably of convex shape and aligned with more than one photovoltaic cell 19′ communicating with more than one pulse transformer 23′, from which the ground electrode 6 extends. As specifically shown, the surgically implanted unit 7 can include an optical lens 18′, three photovoltaic cells 19′ and three pulse transformers 23′. The optical lens 18′, photovoltaic cells 19′ and pulse transformers 23′ are structurally and functionally similar to those described in FIGS. 2 and 4. The optical lens 18′ can be constructed to receive and/or function with pulsed infrared light frequencies corresponding to an acceptable range of audio frequencies, for example between about 125 Hz and about 8,500 Hz, preferably between about 250 Hz and about 4,000 Hz, more preferably between about 500 Hz and about 2,000 Hz, and most preferably about 1,000 Hz, and excluding light frequencies outside of this range. The optical lens 18′ can separate the channels by frequency for use with the multiple channel device implementation. The pulse transformers 23′ are each electrically connected to the active electrode 10 and isolate output a bipolar electrical current through the electrode 10 as interleaved pulsed multichannel electrical fields. The distal end of the electrode 10 preferably includes a multi channel band 13 through which the interleaved pulsed electrical fields are delivered as an electrical signal to the cochlea nerves 11. The ground electrode 6 is electrically connected to each pulse transformer 23′ and provides a similar electrical signal return path as described above.

As particularly depicted in the embodiment shown in FIG. 4, the surgically implanted unit 7 can also include a signal amplifier 20 electrically powered by a power source 21. The signal amplifier 20 is electrically connected between the photovoltaic cell 19 and the pulse transformer 23. Preferably, the signal amplifier 20 increases the amplitude or power of the signal delivered from the photovoltaic cell 19 and received by the pulse transformer 23. The internal power source 21 can be any type including, but not limited to glucose fuel cell, enzymatic biofuel cell, piezoelectric, thermo-voltaic, or other types of energy scavenging power source. As alternatively depicted in FIG. 5, the surgically implanted unit 7 can include at least two, and preferably three, signal amplifiers 20′ electrically powered by the power source 21, and functioning similarly to the signal amplifier described in FIG. 4.

In an additional example, the pulsed infrared cochlear implant system can operate with the embodiments shown in FIGS. 2-5, the transmitted signal can be encoded such that the light is actively transmitted from the light emitting diode to the photocell as an idle state. Thus the implanted unit can harvest power from the photocell during the normally-characterized inactive idle state.

In addition to spatial stimulation and high pulse rates to achieve pitch discrimination and temporal fine structure of sound, pulse width modulation of the light pulses from the pulse transformer 23, 23′ can also be used to enhance pitch discrimination. By utilizing a pulse stimulus rate above the frequency response of the neuron, the pulse width could be varied at a lower frequency within the audible stimulus range—preferably between about 200-500 Hz, in which phase locking auditory sensitivity peaks. Thus, a fundamental pulse rate in the range of 2 to 10 KHz could increase in duration (duty cycle) from less than a micro-second (less than 1% duty cycle) to up to 50%. The rate at which the duty cycle varies is proportional to the frequency of the detected sound; and the amplitude of the duty cycle is proportional to the amplitude of the sound detected. The electrical signals delivered to the spiral ganglion cells are transmitted to the second order auditory neurons and to the brain for integration and interpretation. The duty cycle can be preferably varied from 1% to 80% at a rate proportional to the audible modulating frequency. Thus, the total sensible energy supplied to the auditory nerve can vary at a rate proportional to the frequency of the modulating sound, and the amount of duty cycle variation can be proportional to the amplitude of the modulating sound.

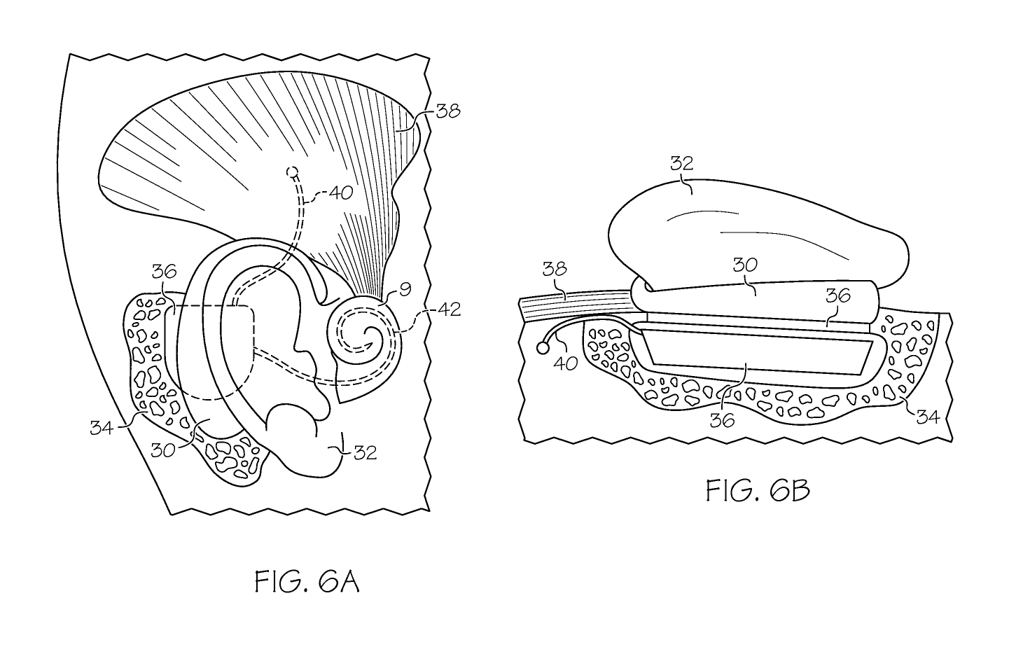

In an alternative embodiment infrared modulation system depicted in FIGS. 6A and 6B, a surgically implanted unit is implanted within the mastoid cavity 34. The surgically implanted mastoid unit includes a ground electrode 40 and a multichannel active electrode 42, each of which function similarly to those in the embodiment described in FIG. 1. The ground electrode 40 is positioned beneath the temporalis muscle 38. The active electrode 42 is inserted through a posterior tympanotomy, or facial recess surgical approach, into the anatomical cochlea 9 through the round window membrane (not shown in FIGS. 6A, 6B), for example through a cochleostomy. The portion of the unit surgically implanted within the mastoid cavity 34 contains a photovoltaic cell receiver 36. A sound processor 30 is positioned on a proximal ear auricle 32. Similarly to the processor described in FIG. 1, the sound processor 30 includes an infrared transmitter and a microphone, which is aligned laterally to the skin and immediately overlying the photovoltaic cell receiver 36 implanted within the mastoid cavity 34. The sound processor 30 infrared transmitter includes LEDs, which transmit a pulsed IR signal across the skin overlying the mastoid cavity 34 to the underlying photovoltaic cell receiver 36. Through the same manner described in the embodiment in FIGS. 1-5, the photovoltaic cell receiver 36 delivers a signal through the active electrode 42 to the cochlea 9.

The alternative embodiment described in FIGS. 6A-6B obviates the need for magnets in the radiofrequency transmission schemes found in traditional transmastoid cochlear implant configurations.

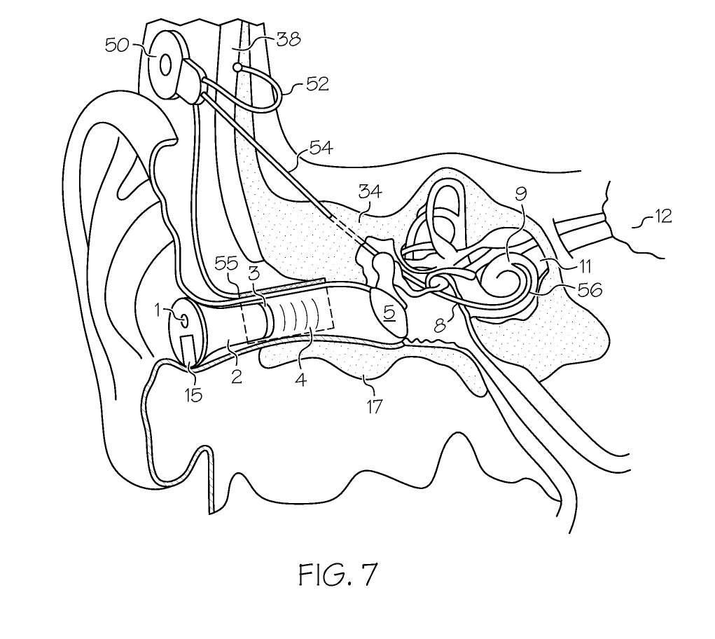

In a second alternative embodiment infrared modulation system depicted in FIG. 7, a surgically implanted unit 50 is implanted within the mastoid cavity 34. The surgically implanted mastoid unit includes a ground electrode 52 and a multichannel active electrode 54 each of which function similarly to those in the embodiment described in FIG. 1. The ground electrode 52 is positioned beneath the temporalis muscle 38. The active electrode 54 is inserted through a posterior tympanotomy, or facial recess surgical approach into the anatomical cochlea 9 through the round window membrane 8, for example through a cochleostomy. The surgically implanted unit 50 includes a photovoltaic cell receiver 55 that is secured between the skin of the external auditory canal 17 and the posterior bony canal wall. The ear processor 2, described in FIG. 1, is similarly positioned within the ear canal 17. The LEDs on the transmitter 3 are oriented such that they transmit the pulsed IR signal 4 through the skin of the external auditory canal 17 to the photovoltaic cell receiver 55. Through the same manner described in the embodiment in FIGS. 1-5, the photovoltaic cell receiver 55 delivers a signal through the active electrode 42 to the cochlea 9.

The second alternative embodiment described in FIG. 7 obviates the need for magnets found in the traditional radiofrequency configurations of transmastoid cochlear implants.

The transmastoid embodiments described in FIGS. 6A-7 can be effectively combined with a hearing aid for EAS application, for example the EAS application described above. Since the transmastoid embodiments described in FIGS. 6A-7 position the photovoltaic cell 36, 55 outside of the middle ear space, the middle ear space is kept clear and allows for more efficient acoustic transmission of a signal through the middle ear mechanism from the processor 2, 30.

In example forms, the invention relates to a novel structural combination of conventional materials and discrete components associated with the aforementioned infrared modulation system. The invention is not limited to the particular detailed configuration thereof. Accordingly, the structure and arrangement of these conventional components have, for the most part, been illustrated in the drawings by readily understandable diagram representations and schematic diagrams. The drawings show only those specific details that are pertinent to the apparatus according to the depicted example embodiment of the present invention in order not to obscure the disclosure with structural details which will be readily apparent to those skilled in the art having the benefit of the description herein. Thus, the schematic diagram illustrations of the Figures do not necessarily represent the mechanical structural arrangement of the exemplary system, and are primarily intended to illustrate major hardware structural components of the apparatus of the present invention in a convenient functional grouping whereby the present invention may be more readily understood.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims, means-plus-function clause is intended to cover the structures described herein as performing the recited function and not only structural equivalents but also equivalent structures. Thus, by way of analogy, although a nail and a screw may not be structural equivalents in that a nail employs a cylindrical surface to secure wooden parts together whereas a screw employs a helical surface, in the environment of fastening wooden parts, a nail and a screw may be equivalent structures.

While the invention has been described with reference to preferred and example embodiments, it will be understood by those skilled in the art that a variety of modifications, additions and deletions are within the scope of the invention, as defined by the following claims.

What is claimed is:

1. A hearing assistance device for use with respect to a mastoid cavity and an ear canal comprising a tympanic membrane, external ear canal bone, incudo-stapedial joint of an ossicular chain and a cochlea having a round window membrane, the hearing assistance device comprising:
a removable unit comprising a microphone to convert a received sound into electrical sound signals, a digital audio signal processor to process the electrical sound signals into envelopes of the electrical sound signals, and an infrared transmitter to transmit the envelopes of sound signals as packages of infrared light pulses; and
an implant unit comprising a photovoltaic cell to receive the packages of light pulses and demodulate the packages of light pulses into electrical signals, and at least one active electrode that delivers the demodulated electrical signals to the cochlea.

2. The hearing assistance device of claim 1, wherein the infrared light pulses are color coded varying by wavelength.

3. The hearing assistance device of claim 1, wherein the photovoltaic cell comprises a plurality of quantum dots.

4. The hearing assistance device of claim 1, wherein the at least one active electrode is configured to deliver the demodulated electrical signals through multiple channels.

5. The hearing device of claim 1, wherein the packages of infrared light pulses are transmitted as an idle state at a variable pulse width of between 200 Hz and 500 Hz, and are transmitted at a pulse rate duration of between 1% duty cycle to 80% duty cycle.

6. The hearing device of claim 1, wherein the implant unit further comprises a ground electrode secured within the bone of the external ear canal, the ground electrode receiving a signal return from the demodulated electrical signals delivered to the cochlea.

7. The hearing device of claim 1, wherein the implant unit further comprises a ground electrode attached to the incudo-stapedial joint of the ossicular chain, the ground electrode receiving a signal return from the demodulated electrical signals delivered to the cochlea.

8. The hearing device of claim 1, wherein the implant unit further comprises a pulse transformer that is configured to isolate the electrical signals from the photovoltaic cell into encoded frequency envelopes and outputs an electrical current through the at least one active electrode as interleaved pulsed multichannel electrical fields.

9. The hearing device of claim 8, wherein the pulse transformer is configured to isolate a direct current bias of the electrical signals into the encoded frequency envelopes.

10. A hearing assistance device for use with respect to a mastoid canal and an ear canal comprising a tympanic membrane, bone and a cochlea having a round window membrane, the hearing assistance device comprising:
a removable unit comprising a microphone to convert a received sound into electrical sound signals, a digital audio signal processor to process the electrical sound signals into envelopes of the electrical sound signals, and an infrared transmitter to transmit the envelopes of sound signals as packages of infrared light pulses; and
an implant unit comprising a photovoltaic cell to receive the packages of light pulses and demodulate the packages of light pulses into electrical signals, and a pulse transformer that isolates the electrical signals from the photovoltaic cell into encoded frequency envelopes and outputs an electrical current to the cochlea as interleaved pulsed electrical fields.

11. The hearing device of claim 10, wherein the pulse transformer is configured to isolate a direct current bias of the electrical signals into the encoded frequency envelopes.

12. The hearing device of claim 11, wherein the electrical current output is bipolar.

13. The hearing device of claim 11, wherein the implant unit further comprises at least one active electrode that delivers the interleaved pulsed electrical fields to the cochlea.

14. The hearing assistance device of claim 13, wherein the at least one active electrode is configured to deliver the demodulated electrical signals through multiple channels.

15. The hearing device of claim 13, wherein the at least one active electrode extends within the cochlea.

16. The hearing device of claim 13, wherein the implant unit is implanted within the mastoid cavity.

17. The hearing assistance device of claim 10, wherein the infrared light pulses are color coded varying by wavelength.

18. The hearing device of claim 10, wherein the implant unit further comprises a ground electrode secured within the cochlea bone, the ground electrode receiving a signal return from the demodulated electrical signals delivered to the cochlea.

19. The hearing device of claim 10, wherein the pulse transformer isolates a direct current bias of the electrical signals into the encoded frequency envelopes.

20. The hearing device of claim 10, wherein the removable unit and the implant unit are positioned on opposing sides of the tympanic membrane.

21. A method for delivering an audio sound signal to the nerves extending from a cochlea, the method comprising:
converting a received audio sound signal into a plurality of electrical signals;
processing the plurality of electrical signals into envelopes of electrical sound signals;
transmitting the envelopes of sound signals as packages of infrared light pulses;
demodulating the packages of infrared light pulses into electrical signals;
isolating the demodulated electrical signals into encoded frequency envelopes; and
outputting the encoded frequency envelopes as interleaved pulsed electrical fields within the cochlea.

22. The method of claim 21, wherein the received audio sound signal is converted into a plurality of electrical signals by a digital signal processor.

23. The method of claim 21, wherein the envelopes of sound signals are transmitted as packages of infrared light pulses by a Light Emitting Diode.

24. The method of claim 21, wherein the packages of light pulses are demodulated into electrical signals by a photovoltaic cell.

25. The method of claim 24, further comprising:
encoding the envelopes of sound signals to be actively transmitted as an idle state; and
harvesting power from the photocell during inactive transmission.

26. The method of claim 25, wherein the envelopes of sound signals are transmitted as an idle state at a variable pulse width of between 200 Hz and 500 Hz, and are transmitted at a pulse rate duration of between 1% duty cycle to 80% duty cycle.

27. The method of claim 21, wherein the demodulated electrical signals are isolated into encoded frequency envelopes by a pulse transformer.

28. The method of claim 21, wherein the encoded frequency envelopes are outputted as interleaved pulsed multichannel electrical fields by at least one active electrode.

29. The method of claim 21, wherein the infrared light pulses are color coded varying by wavelength.

30. The method of claim 21, wherein the packages of infrared light pulses are passively demodulated into electrical signals.
